(19) United States
(12) Patent Application Publication
(10) Pub. No.: US 2020/0245949 A1
(43) Pub. Date: Aug. 6, 2020

(54) Forecasting Mood Changes from Digital Biomarkers

(71) Applicant: Mindstrong Health, Palo Alto, CA (US)

(72) Inventor: Paul Dagum, Los Altos Hills, CA (US)

(21) Appl. No.: 16/265,475

(22) Filed: Feb. 1, 2019

(51) Int. Cl. A61B 5/00; G16H 50/50; A61B 5/16; A61B 5/11

(52) U.S. Cl. CPC A61B 5/7275; G16H 50/50; A61B 5/165; A61B 5/6898; A61B 5/1124; A61B 5/162; A61B 2503/08; A61B 5/7435; A61B 5/7475; A61B 5/747; A61B 5/1118; A61B 5/1123; A61B 2562/0219; A61B 5/4803

(57) ABSTRACT

The present invention extends to methods, systems, and computer program products for forecasting mood changes from digital biomarkers and more generally for forecasting changes in a neuropsychological clinical assessment. User interaction data indicative of a user's interaction with a mobile device is passively captured over a period of time. A function mapping (modeling a neuropsychological test for a brain health metric) is executed to compute a digital biomarker for a brain health metric from the captured user interaction data. A prior digital biomarker for the brain health metric (computed from previously captured user interaction data by executing the function mapping) is accessed. A difference is detected between the digital biomarker and the prior digital biomarker. A change in a score of the neuropsychological test score is forecast to occur within a specified time range in the future based on the detected differences. The forecast change is output.

### CROSS-REFERENCE TO RELATED APPLICATIONS

Not Applicable

### BACKGROUND

### 1. Field of the Invention

The invention relates generally to measuring cognition and mood and, more specifically, to forecasting changes in cognition and mood from digital biomarkers.

### 2. Related Art

Cognitive function tests measure a person's cognitive abilities across a broad range of cognitive domains such as memory (working memory, semantic memory, episodic memory), attention, processing speed (visuospatial, symbol substitution), verbal skills, general intelligence, and executive function. Cognitive function tests typically administered by a trained psychometrician requiring several hours of testing. Based at least in part on the need for a skilled administrator and the length of testing, cognitive function tests are performed relatively infrequently (e.g., once a year).

Scores on cognitive function tests for the same individual can vary for a variety of reasons. For example, test scores can vary due to a change in the person's cognitive function. However, scores can also vary due to the subjective nature of the interpretation of the tests or due to situational factors that may affect an individual on the day of the test. When tests scores vary due to interpretation and/or situational factors, the accuracy of measuring a person's cognitive function using a cognitive function test reduced.

Brain health is critical to our success as individuals in an increasingly cognitive demanding society. In school aged children and adolescents, brain health is responsible for academic success. In working individuals, brain health leads to improved job performance, and in the elderly it enables autonomy, independence and greater enjoyment from activities.

Cognitive function is a measure of brain health, and factors that affect the brain also affect cognitive function. These factors can be categorized into situational, traumatic, and disease related. Situational factors include lifestyle decisions on diet, social engagement, intellectual stimulation, physical activity, sleep patterns, and stress levels. Situational factors can have a shorter term affect and can change relatively frequently. During periods of high stress, poor sleep, and inadequate physical activity (e.g., when working on an important and time-consuming work project), a person will perform worse on a cognitively demanding task. However, once stress is reduced, sleep improves, and physical activity increases (e.g., when the work project is completed), the person will perform better on the cognitively demanding task

Unfortunately, there is no known reliable system or method for repeated and regular assessment of cognitive function to inform a person of the harm or benefit that current lifestyle decisions have on their brain health. Repeat cognitive function testing by a psychometrician is neither practical nor reliable when repeated more frequently than once per year because the individual acquires test-taking skills for the test. Similarly, the emergence of online tests available through many application vendors have documented deficiencies. For example, subjects develop test taking skills that increase their scores but that do not transfer well to real world activities and undermine the test's sensitivity and specificity to cognitive changes.

Traumatic factors affecting cognitive function include blunt or penetrating head injuries. Unlike situational factors, the effect of traumatic brain injury on cognitive function is not generally reversible. Traumatic brain injury is increasingly recognized as a contributor to cognitive function deficits in players of contact sports. Early detection of changes in cognitive function for contact sport athletes is paramount to their brain health and requires a systemic method to measure cognitive function that is repeatable, reliable, and unobtrusive.

Many diseases are known to affect brain health and cognitive function. The progression of aging into mild cognitive impairment and Alzheimer's disease is of great societal concern because of its rapidly increasing prevalence in an increasingly older society. Prior to developing Alzheimer's disease, patients go through a six-year prodromal phase of cognitive decline. The societal burden of mental disease in the elderly is staggering and poised to worsen.

Monitoring cognitive decline with yearly cognitive function tests is sub-optimal. A person can potentially go months being unaware of a diagnosable (and possibly treatable) reduction in brain health and cognitive function. Further, due to interpretation and/or situational factors, cognitive function test results may not fully indicate a reduction in brain health and cognitive function.

Mood disorders include depressive disorders, bipolar disorders, and substance-induced mood disorders. They affect people of all ages and impair multiple cognitive domains. Mood disorder therapy focuses on the behavioral and mood related facets of the disease to the detriment of the cognitive function deficits. Medications to treat mood disorders can worsen cognitive domains such as memory. The impact of cognitive deficits in a person's school or job performance can be significant, is exacerbated by treatment, and can go unrecognized due to infrequent cognitive function testing.

Many other diseases are known to affect brain health and cognitive function. These include neurovascular disorders including multi-infract dementia, hepatic failure with encephalopathy, renal failure, congestive heart failure, and various infectious disease and viral illness to name a few. Individuals with any of these disorders are at risk for cognitive impairment.

### BRIEF DESCRIPTION OF THE DRAWINGS

The specific features, aspects and advantages of the present invention will become better understood with regard to the following description and accompanying drawings where:

FIG. 1 illustrates an example block diagram of a computing device.

FIG. 2 illustrates an example electronic device architecture that facilitates passively capturing user interaction data at electronic device.

FIG. 3A illustrates an example electronic device architecture that facilitates forecasting a mood change.

FIG. 3B illustrates an example computer architecture that facilitates forecasting a mood change.

FIG. 4 illustrates a flow chart of an example method for forecasting a mood change.

### DETAILED DESCRIPTION

The present invention extends to methods, systems, and computer program products for forecasting mood changes from digital biomarkers

Measurement-based care is an important priority for improving outcomes in psychiatry. While various self and observer ratings of depression are available, neither patients nor providers seem willing to adopt these for the long-term management of mood disorders. Current assessments of mood require the burden of time and effort, are episodic providing a sparse snapshot, and are usually completed in clinical rather than ecological settings. They also lack the predictive or clinical value of biomarkers we have come to expect in the rest of medicine.

There is currently no clinically-actionable biomarker for depression analogous to HbA1c for diabetes or blood pressure for hypertension. However, the advent of smartphones, which globally are used intensively and extensively, may provide an opportunity for a different kind of biomarker. Digital phenotyping includes the use of digital features from smartphones or wearables to monitor cognition, mood, and behavior. Digital biomarkers created from a person's touch-screen interactions, or more generally human-computer interactions, with a smartphone can predict test scores of cognitive function. These digital features have several desirable properties. They are objective measures based on thousands of daily touchscreen events. They are ecological and passive, providing daily observations from routine phone use and facilitate immediate global scale to billions of people with smartphones.

Accordingly, digital biomarkers can also be used to assess mood and cognition in patients being treated for mood disorders and/or that are at risk for mood disorders, such as, for example, depression (e.g., Major Depressive Disorder (MDD) or Bipolar Depression (BD)), anxiety, mania or psychosis. Cycles of mood fluctuation can be used to identify digital biomarkers correlated with scores obtained from (e.g., gold-standard) clinician-administered assessments of mood and cognition. Further, time-series of daily digital biomarkers can be used to forecast future changes in clinical assessments.

In more detail, the introduction of mobile devices and their broad adoption has revolutionized how society interacts both with each other and with their surroundings. A smartphone today enables a user to make calls, send and receive emails and text messages, find their location on a map or retrieve directions to a destination point, browse the internet, download and play game applications, and a host of other activities. In addition, these smartphones are equipped with accelerometers and gyroscopes that sense the device's acceleration and orientation in 3-dimensions. Processing of the acceleration and orientation signals reveals a user's activity such as whether the person is walking or jogging. Other electronic devices including tablets and wearable electronic devices, such as, watches, smart clothing, and glasses are also capable of delivering much of the functionality found in a smartphone.

In one aspect, an application is installed on a mobile phone or other electronic device. The application passively captures user interaction data indicative of a user's interaction with the mobile phone or other electronic device. User interaction data can be captured during multiple user encounters over a period of time. The application can capture interaction data without capturing any content information. Machine learning algorithms can be used to create digital biomarkers from the captured interaction data and that are correlated with standard clinical assessments of mood. Digital biomarkers can be created on a daily basis. Changes in biomarkers can be used to predict mood changes as measured by changes in the clinical assessments of mood three to ten days prior to occurrence. As such, digital biomarkers can serve as an early signal of patients at risk for relapse.

Analyzing user interaction data can include identifying and extracting event patterns from the user interaction data. A pattern can include two successive events, such as, for example, tapping on the space-bar followed by the first character of a word. Patterns can be collected to a specific use context. For example, tapping on a character followed by another character could be collected at the beginning, middle, and end of a word.

Patterns can correspond to tasks that are repeated up to several hundred times per day by a user during normal usage of an electronic device, such as, a mobile phone. For each type of pattern, a time-series composed of the time interval between patterns can be generated. The time-series can be further segmented into daily time-series. Mathematical transforms can be applied to each daily time-series to produce (e.g., hundreds) of distinct daily features from a single digital pattern, and combined in novel ways to create a digital biomarker that correlates with a clinical assessment of cognition or mood. Various statistical, machine learning, and modeling techniques can be used to identify the combination of digital pattern features that correlate with clinical assessments of mood and/or cognition. In one aspect, an extension of supervised kernel Principal Component Analysis (PCA) is used in combination with leave-one-out cross-validation (LOOCV) or out-of-bag prediction error using bootstrap aggregation to combine digital patterns to produce digital biomarkers that correlate with clinical assessments of mood.

An individual (e.g., person 211) can be clinically assessed. A clinical assessment can include neuropsychological tests for one or more of: depression, anxiety, mania or psychosis. A clinical assessment can also include neuropsychological tests for one or more of: memory, processing speed, executive function, language, dexterity or intelligence.

A clinical assessment can include observer-rated and/or self-rated measures. Observer-rated assessments include Hamilton Depression Rating Scale (Ham-D), Hamilton Anxiety Rating Scale (Ham-A), Smith-Hamilton Pleasure Scale (SHAPS), Clinical Global Impression of Severity (CGI-S) to measure anhedonia, Clinical Global Impression of Severity (CGI-S), and Positive and Negative Syndrome Scale (PANSS), etc. Initial ratings can be determined face-to-face with follow up ratings completed by phone. Self-rated assessments include Patient Health Questionnaire 9 (PHQ-9) with modified instructions to probe for the previous 24 hours as opposed to previous 14 days.

Self and observer ratings can be determined at designated intervals following administration of medication, such as, ketamine or interventions such as repeated transcranial magnetic stimulation (rTMS). For example, assessments can be collected at 1, 2, 3, 7, 10, 14, 17 and 21 days for Ham-D and Ham-A and weekly for SHAPS and CGI-S. After 21 days, all assessments can be collected weekly until the medication or intervention is administered again due to relapse. Relapse can be defined apriori. In one aspect, relapse is defined as a PHQ-9 score greater than a specified value, such as, for example, 20. In another aspect, relapse is defined as a percentage reduction in PHQ-9 improvement from pre-treatment with medication (e.g., ketamine) to post treatment. For example, in the PHQ-9 score changed from 18 pre-medication to 10 post-medication, relapse can be defined as a PHQ-9 score of 16 (i.e., the sum of 10 and (0.75× the 8-point improvement).

Ketamine or rTMS is capable of producing rapid (e.g., within 24 hours) and robust antidepressant effects. However, the antidepressant effects are relatively short lived (days to weeks). As such, treatment of depression with ketamine or rTMS, for example, requires repeated administration and results in cycles of mood improvement followed by relapse over 1-6 weeks. These cycles of mood fluctuation can be used to identify digital biomarkers correlated with the scores obtained from observer-rated and/or self-rated measures.

The individual installs a capture module (e.g., capture module 209) on his or her electronic device. In general, the capture module captures user interaction data daily over multiple episodes of recovery and relapse. The capture module can passively capture electronic device (e.g., smart phone) features associated with and predictive of clinical ratings of mood, anxiety, anhedonia, depression clinical severity, and psychosis symptom severity. As such, various statistical, machine learning, and modeling techniques can be used to generate: (a) panels of biomarkers having high correlations with clinical ratings (Ham-D, Ham-A, SHAPS, CGI-S, PANSS etc.) and (b) a separate set of biomarkers predictive of the same clinical ratings (and thus providing a forecast of recovery and relapse).

Data capture can begin after clinical assessment and at least one day prior to an initial administration of ketamine or rTMS (or some other medication or intervention for treating depression). After clinical assessment, the individual may be diagnosed with depression. Thus, the capture module can capture user interface data for at least one day pre-medication (or when the user is “depressed”). Biomarkers associated with the user being “depressed” can be derived from the user interaction data captured pre-medication.

The capture module can continue to capture user interaction data post-medication. Biomarkers associated with the user under the antidepressant effects of the medication or intervention can be derived from user interaction data captured post-medication or post-intervention. Post-medication/post-intervention biomarkers can be compared to pre-medication biomarker/pre-intervention to identify differences and quantify the effect of the medication/intervention.

The capture module can continue to capture user interaction data even after the antidepressant effects of the medication/intervention treatment are realized. New biomarkers can be derived each day from user interaction data captured that day. Each day, the new biomarkers can be compared to the pre-treatment biomarkers and the post-treatment biomarkers (and possibly also biomarkers from other days) to determine if the individual is relapsing and/or to predict when the individual is likely to relapse.

In another aspect, input of a supervised kernel Principal Component Analysis (PCA) is pre-processed. Features from the digital patterns that satisfy a specified False Discovery Rate (FDR) are used. For targets, a change in digital patterns from the day prior to an individual's clinical assessment relative to digital patterns from the day of the clinical assessment are computed. A daily change in target is normalized to arrive at a daily change in the target by dividing the number of elapsed days between two clinical assessments. Using this pre-processed input for supervised kernel Principal Component Analysis (PCA) together with (LOOCV) or out-of-bag prediction forecasts for daily target changes can be generated. Forecasts can then be extrapolated to a target clinical assessment at some time, for example, three to ten days, in the future.

As such, a forecast of clinical change in mood that correlates with the measured change from repeated clinical assessments can be used to create a predictive test of clinical change that does not require the repeated clinical assessments. For each mood assessment, the number of forecasts that did or did not exceed an observed clinical change were computed at varying thresholds. From the number of forecasts, true positive, false positive, true negative, and false negative rates can be calculated at each threshold. Using these rates, the sensitivity, specificity, and receiver-operator curve (ROC) can be calculated over the range of thresholds. Positive and negative predictive values can also be calculated.

In one aspect, the supervised kernel Principal Component Analysis (PCA) selects a specified number of digital patterns, such as, between 5 and 15 digital patterns, that pass a strict FDR. Using the temporal changes in these patterns as prospective digital biomarkers in the supervised kernel Principal Component Analysis (PCA) to learn the prospective digital biomarkers that are most predictive of the observed changes clinical assessments, changes in clinical assessments can be predicted in out-of-sample data. For example, the daily changes in multiple digital patterns together can be used to learn changes in clinical assessments and predict a next clinical rating occurring at 3 to 10 days in the future. However, clinical ratings can be predicted in other day ranges as well, such as, for example, 1 to 12 days in the future, 2 to 11 days in the future, etc. Accordingly, these forecasting and predictive digital biomarkers are clinically relevant.

In this description and the following claims, “digital biomarker” is defined as a group of digital patterns and their features which may have clinical utility for either monitoring or predicting clinical state.

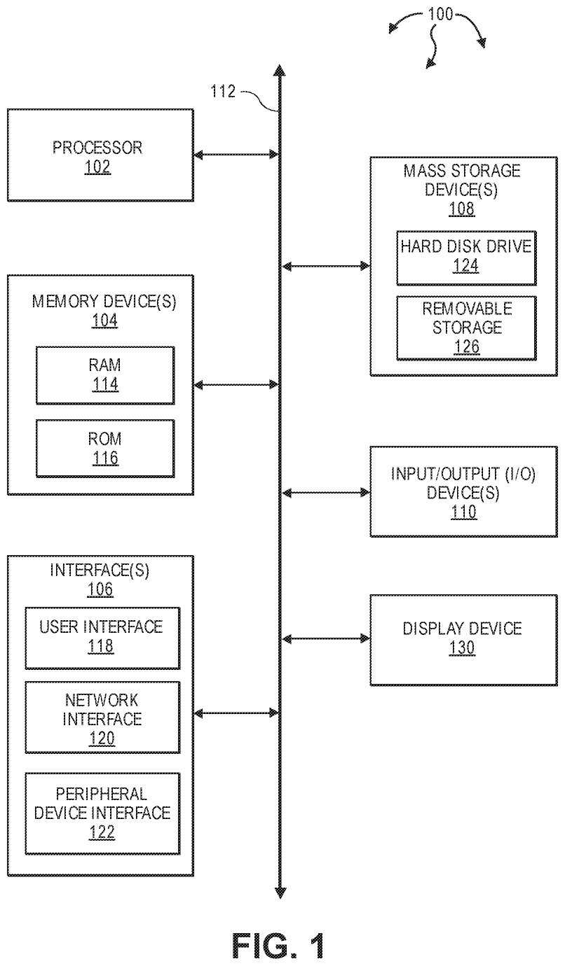

FIG. 1 illustrates an example block diagram of a computing device 100. Computing device 100 can be used to perform various procedures, such as those discussed herein. Computing device 100 can function as a server, a client, or any other computing entity. Computing device 100 can perform various communication and data transfer functions as described herein and can execute one or more application programs, such as the application programs described herein. Computing device 100 can be any of a wide variety of computing devices, such as a mobile telephone or other mobile device, a desktop computer, a notebook computer, a server computer, a handheld computer, tablet computer and the like.

Computing device 100 includes one or more processor(s) 102, one or more memory device(s) 104, one or more interface(s) 106, one or more mass storage device(s) 108, one or more Input/Output (I/O) device(s) 110, and a display device 130 all of which are coupled to a bus 112. Processor(s) 102 include one or more processors or controllers that execute instructions stored in memory device(s) 104 and/or mass storage device(s) 108. Processor(s) 102 may also include various types of computer storage media, such as cache memory.

Memory device(s) 104 include various computer storage media, such as volatile memory (e.g., random access memory (RAM) 114) and/or nonvolatile memory (e.g., read-only memory (ROM) 116). Memory device(s) 104 may also include rewritable ROM, such as Flash memory.

Mass storage device(s) 108 include various computer storage media, such as magnetic tapes, magnetic disks, optical disks, solid state memory (e.g., Flash memory), and so forth. As depicted in FIG. 1, a particular mass storage device is a hard disk drive 124. Various drives may also be included in mass storage device(s) 108 to enable reading from and/or writing to the various computer readable media. Mass storage device(s) 108 include removable media 126 and/or non-removable media.

I/O device(s) 110 include various devices that allow data and/or other information to be input to or retrieved from computing device 100. Example I/O device(s) 110 include cursor control devices, keyboards, keypads, barcode scanners, microphones, monitors or other display devices, speakers, printers, network interface cards, modems, cameras, lenses, radars, CCDs or other image capture devices, and the like.

Display device 130 includes any type of device capable of displaying information to one or more users of computing device 100. Examples of display device 130 include a monitor, display terminal, video projection device, and the like.

Interface(s) 106 include various interfaces that allow computing device 100 to interact with other systems, devices, or computing environments as well as humans. Example interface(s) 106 can include any number of different network interfaces 120, such as interfaces to personal area networks (PANs), local area networks (LANs), wide area networks (WANs), wireless networks (e.g., near field communication (NFC), Bluetooth, Wi-Fi, etc., networks), and the Internet. Other interfaces include user interface 118 and peripheral device interface 122.

Bus 112 allows processor(s) 102, memory device(s) 104, interface(s) 106, mass storage device(s) 108, and I/O device(s) 110 to communicate with one another, as well as other devices or components coupled to bus 112. Bus 112 represents one or more of several types of bus structures, such as a system bus, PCI bus, IEEE 1394 bus, USB bus, and so forth.

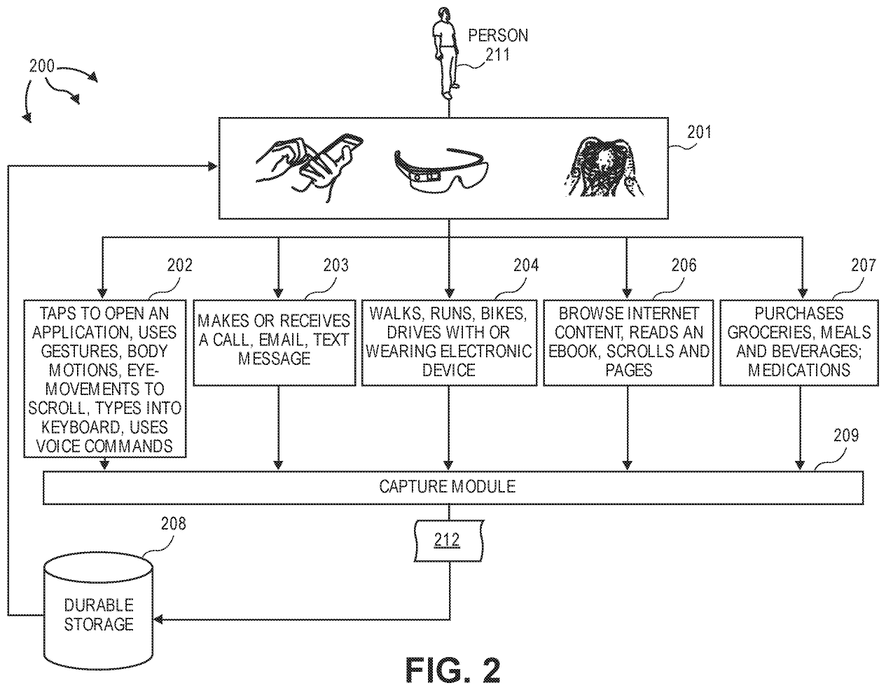

FIG. 2 illustrates an example electronic device architecture 200 that facilitates passively capturing user interaction data at electronic device. Person 211 interacts with his or her electronic device 201 in any of a variety of different ways. Electronic device 201 can be a smart phone, tablet computer, a wearable electronic device (e.g., electronic glasses, electronic wristband device, smart clothing, etc.), a household electronic device (e.g., desktop computer, remote control, etc.), etc.

Person 211 can interact with applications and other modules at electronic device 201 through various gestures. Gesture activity 202 represents person 211's gestures performed to interact with applications and other modules at electronic device 201. Gesture activity 202 can include person 211 (a) tapping a touch screen, speaking, using a touch pad, or invoking an external device, such as, mouse to open an application, (b) using touch-screen gestures, eye movements, body motions including head tilting and swiping, or invoking an external device, such as, mouse to scroll within an application, (c) typing messages, directions, and other content on a keyboard exposed by the application or uses voice commands to do the same, (d) reading and scrolling through content, (e) making calls or (f) listening to messages. Capture module 209 can capture gesture activity 202 and store gesture activity 202 in durable storage 208.

Person 211 can send communication to others using electronic device 201. Communication activity 203 represents person 211's communication related interaction through input/output components of electronic device 201. Communication activity 203 can include person 211 making or receiving phone calls, sending or receiving email messages, or sending or receiving text messages. Communication activity 203 can also include communication frequency and various qualitative characteristics of communication activity. Capture module 209 can capture communication activity 203, including the date and time of making or receiving phone calls, sending or receiving email messages, or sending or receiving text messages, the duration of calls, sender and recipient phone numbers, emails address, etc. Capture module 209 can store communication activity 203 in durable storage 208.

Person 211 can interact with the Internet through electronic device 201. Internet activity 206 represents person 211's interaction with the Internet through input/output components of electronic device 201. Internet activity 206 can include person 211 (a) browsing URLs on an internet-browser application resident on the electronic device or (b) reading an e-book on an e-book reader resident on the device. Internet activity 206 can also include URLs browsed and/or the pages of the e-book read and the start time and end time between URLs and pages. Capture module 209 can capture Internet activity 206 and store Internet activity 206 in durable storage 208.

Capture module 209 can include an intelligence layer. The intelligence layer can derive correlations 212 that correlate gesture activity 202 with communication activity 203, motion activity 204, and Internet activity 206. In this way, capture module 209 can corelate gestures with browsing, paging, scrolling, making calls, reading emails, etc. Capture module 209 can store correlations 212 in durable storage 208.

Capture module 209 can capture application interaction including keyboard inputs, mouse inputs, voice, gestures, body motions, eye movements, touch-screen gestures, such as tapping and swiping. For applications running on wearable electronic devices, the capture module can capture body motion, such as, head tilting, swiping, or eye movements. The capture modules can capture the date-time of the gesture, body motion or eye movement, the duration and latency, the application in use, and the application view change that results from the gesture, body motion, or eye movement. The capture module can store this user interaction data to durable storage (e.g., 208).

Capture module 209 can capture keyboard entries for applications supporting a keyboard entry mode. Keyboard entries can include alphanumeric entries, backspace entries, capitalization, formatting entries, and editing entries. Capture modules can capture the latency and duration of each keyboard entry, the application in use, and the application view change resulting from the key entry. The capture module can store this user interaction data to durable storage (e.g., 208).

Capture module 209 can capture words and phrases during phone conversations and voice-input commands. The capture module can further capture the application in use, and the application view change resulting from a voice command. The capture module can store this user interaction data to durable storage (e.g., 208).

Capture module 209 can capture sensor data including gyroscope and accelerometer readings from the electronic device, including wearable devices. The sensor data can include information related to user activity and kinetic information including motion, gait, and posture. The capture module can also capture output from a wearable electroencephalogram device. The capture module can store this user interaction data to durable storage (e.g., 208).

Capture module 209 can capture heart rate, blood pressure, pulse oximetry, body temperature, and other physiological data, and associated time stamps from peripheral accessories. The peripheral accessories can be used to obtain biological vital signs of an individual, which can be used to determine if a change (decline) in mood or in cognition is due to a vital sign, such as, fatigue or low blood-glucose levels rather than an actual change (decline) in mood or in cognition. The biological vitals can also be used as an alert of a biological trend possibly having a long-term negative impact on cognitive function, such as hypertension. The capture module can store this user interaction data to durable storage (e.g., 208).

Capture module 209 can capture and time stamp caloric input, nutritional content, alcohol, caffeine consumption as well as medications taken, quantities and dosages. The dietary information can be used as an alert of a dietary trend possibly having an effect on mood and cognition or a long-term negative impact on cognitive function, such as, high alcohol intake or high fat or cholesterol intake. The medication information may be used to determine the negative or beneficial effect that medications and dosages have on mood and cognitive function. The capture module can store this user interaction data to durable storage (e.g., 208).

In one aspect, captured user interaction data stored at durable storage 208 is transmitted to cloud computing resources. Transmission preferably uses a secure channel and can use hypertext transfer protocol secure (HTTPS) protocol to securely transfer the data.

In general, captured user interaction data can be analyzed to detect and/or predict mood changes and/or cognitive changes in an individual. In one aspect, an electronic device (e.g., 201) analyzes user interaction data locally to detect and/or predict mood changes and/or cognitive changes in an individual (e.g., person 211). In another aspect, cloud computing resources analyze user interaction data received from an electronic device (e.g., 201) to detect mood changes and/or cognitive changes in an individual (e.g., person 211).

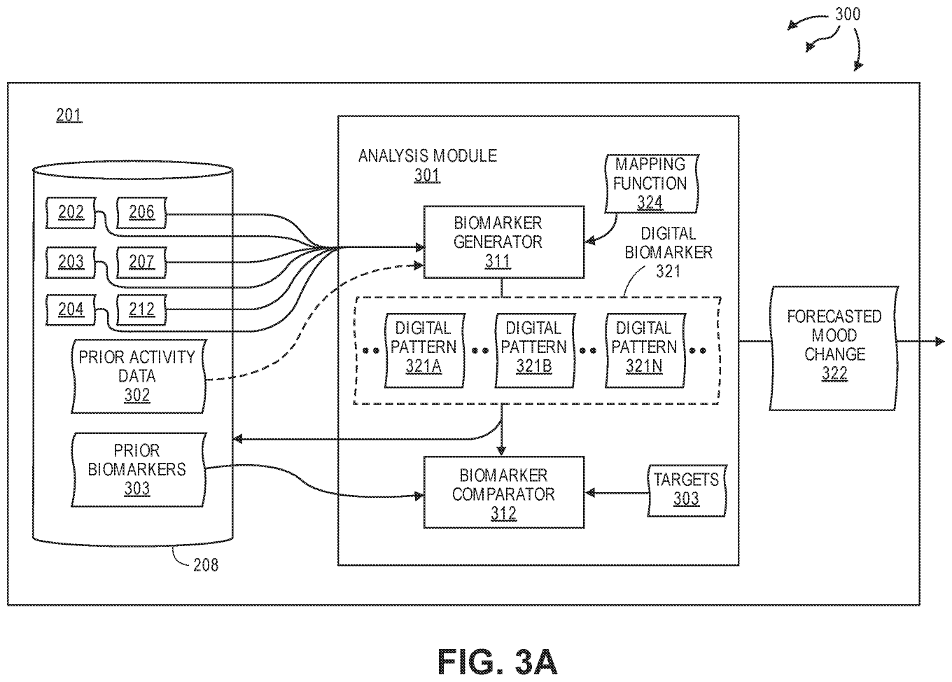

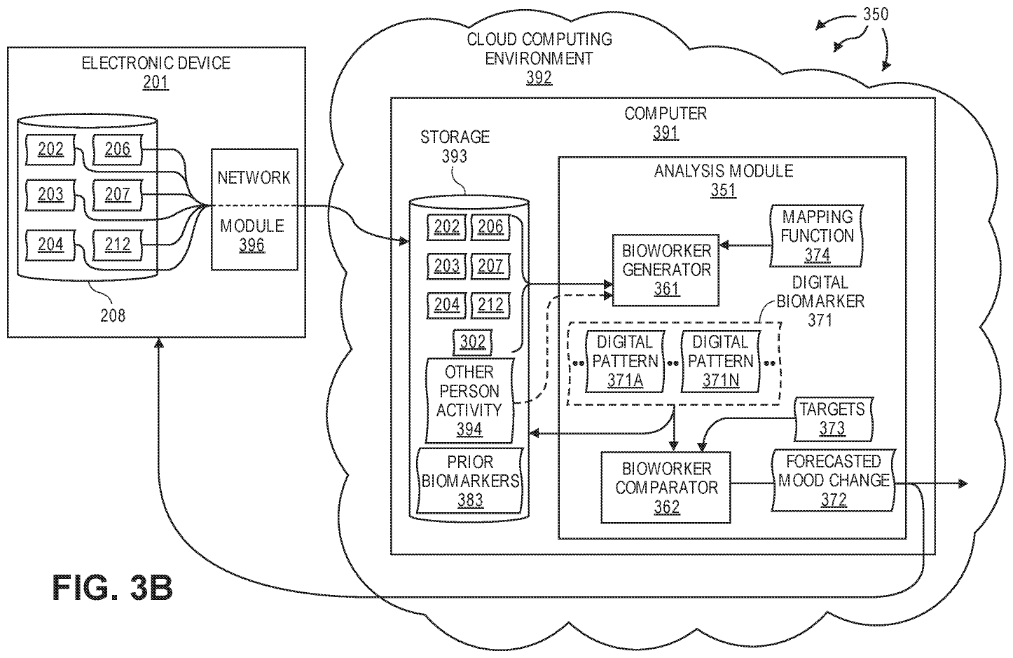

FIG. 3A illustrates an example electronic device architecture 300 that facilitates forecasting a mood change. As depicted in electronic device architecture 300, electronic device 201 includes durable storage 208 and analysis module 301. Analysis module 301 further includes biomarker generator 311 and biomarker comparator 312.

In generally, analysis module 301 can analyze user interaction data including identifying and extracting event (digital) patterns from the user interaction data. A pattern can include two successive events, such as, for example, tapping on the space-bar followed by the first character of a word. Patterns can be collected to a specific use context. For example, tapping on a character followed by another character could be collected at the beginning, middle, and end of a word.

Features of a pattern can include, for example, timing, duration, frequency and amplitude, and number of occurrences. The attributes of each pattern include information relating to the when, where, what, and why of each pattern. This data includes, for example, date-time of occurrence, GPS readings, accelerometer and gyroscope readings, physiology and kinetic readings, app in use at the time.

A variety of different event patterns can be identified in and extracted from user interaction data. Application interaction patterns, including recurring combinations of applications opened and closed by a user, frequency and latencies between opening and closing can be identified and extracted. Gesture patterns in a user's gestures, tapping and body motions such as head-tilting, swiping, or eye movements that can be used as commands to an electronic device can be identified and extracted. Patterns in false positive rates, scrolling during search and paging during browsing can be identified and extracted.

Input patterns in keys pressed on a keyboard or other input form-factor and in combination with gesture, tapping, body motion and eye movement inputs can be identified and extracted. For character inputs, recurring keystroke combinations, recurring spelling mistakes, omissions, back-space corrections, irregular latency variances in common words are preferably extracted. Features of each input pattern including duration, frequency, latency, force used, length of messages, and message coherence may be identified and extracted.

Voice patterns in voice commands, words, and phrases used can be identified and extracted. Following signal processing of the voice, recurring combinations of phones and phoneme may be extracted. Features of each voice pattern including voice pitch, amplitude, and frequency spectrum can be identified and extracted. Physical characteristic patterns in electroencephalogram (EEG), locomotion, gait, and posture recorded from wearable electronic devices can be identified and extracted. Features of each physical characteristic pattern derived from the EEG, accelerometer, gyroscope, and other sensor recordings can also be identified and extracted.

Physiologic patterns in heart rate, blood pressure, body temperature, blood oxymetry and other physiologic measurements can be identified and extracted. Together with accelerometer and gyroscope data, a rapid heart rate from anxiety or illness can be distinguished from exercise-induced changes. Features of each physiologic pattern including maximum and minimum measurements, duration of pattern, frequency of pattern, can also be identified and extracted.

Nutritional patterns in recorded daily caloric intake, nutritional intake by food group or food type, intake of alcohol, caffeine, medications including prescription and non-prescription can be identified and extracted. Features such as time of day, total intake, and location of intake can be identified and extracted.

The what, where, when, and why attributes identified and extracted for each pattern may be used to filter those patterns prior to digital pattern generation and biomarker generation. For example, including time of day or day of week may explain variance that can be attributed to individual fatigue and other factors that have negative effects on mood. GPS, gyroscope and accelerometer data can be used to correct for motion artifact from activities such as driving or walking. Further, the physiologic measurements of heart-rate, blood pressure, blood oxymetry, body temperature, and electroencephalogram can be used to correct for the negative effects that physical illness and fatigue have on mood.

In one aspect, biomarker generator 311 executes a mapping function modeling a neuropsychological test (e.g., Ham-D, Ham-A, SHAPS, CGI-S, PHQ-9, etc.) for the brain health metric. The mapping function can incorporate a combination of statistical, machine learning, and modeling techniques to identify and extract activity patterns from user interaction data and generate digital patterns aggregated into a digital biomarker. The mapping function may have been learned through training on other sets of user interaction data from many different users over the course of recovery and relapse of a mental health condition (e.g., depression).

The mapping function can generate a variety of statistics from identified and extracted activity patterns and corresponding features. The computed statistics may also be computed on all available values of the feature or transformed feature, or they may be computed on a subset of values filtered by one or more attributes. The statistics computed may include, for example, mean, median, mode, variance, kurtosis, moments, range, standard deviation, quantiles, inter-quantile ranges, and distribution parameters under different distributions such as exponential, normal, or log-normal.

Biomarker comparator 312 is configured to compare a newly generated digital biomarker to one or more previously generated biomarkers and/or previously computed biomarker targets. From the comparisons, analysis module 301 can detect and/or predict mood changes and/or cognitive changes in an individual. When appropriate analysis module 301 can alert a mental health provider to screen the individual for a predicted clinical change based on the detected differences. The predicted clinical change can be forecast to occur within some time range in the future, such as, for example, within three to ten days after generation of the newly generated digital biomarker.

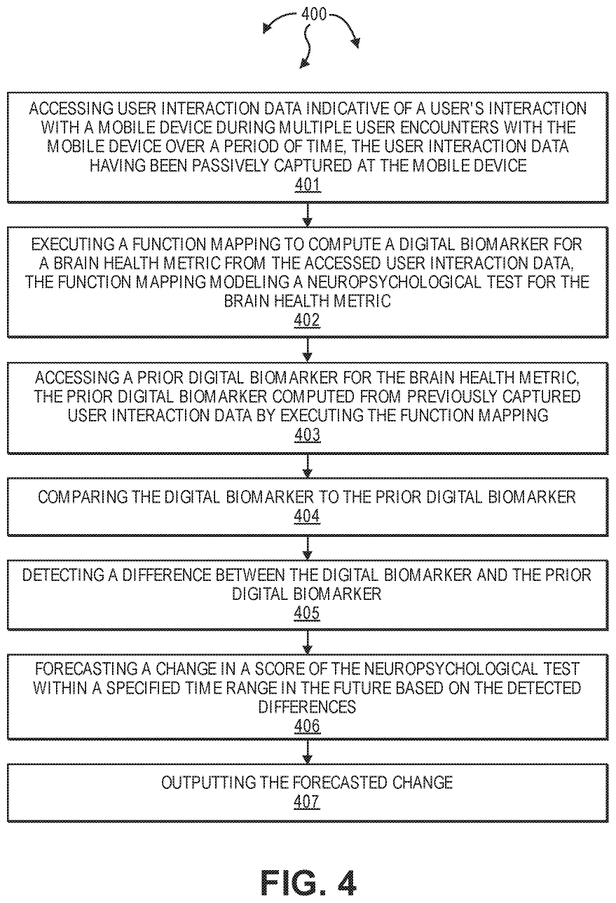

FIG. 4 illustrates a flow chart of an example method 400 for forecasting a mood change. Method 400 will be described with respect the components and data in computer architecture 300.

Method 400 includes accessing user interaction data indicative of a user's interaction with a mobile device during multiple user encounters with the mobile device over a period of time, the user interaction data having been passively captured at the mobile device (401). For example, biomarker generator 311 can access gesture activity 202, communication activity 203, motion activity 204, Internet activity 206, bar code/medical activities 207, and correlations 212 from durable storage 208. Biomarker generator 311 can also access prior activity data 302. Prior activity data 302 can be user interaction data of person 211 that was previously captured and used to generate one or more prior biomarkers.

Method 400 includes executing a function mapping to compute a digital biomarker for a brain health metric from the accessed user interaction data, the function mapping modeling a neuropsychological test for the brain health metric (402). For example, biomarker generator 311 can execute mapping function 324 to compute digital patterns 321A, 321B, . . . , 321N aggregated into digital biomarker 321. Mapping function 324 can model a neuropsychological test for the brain health metric

Method 400 includes accessing a prior digital biomarker for the brain health metric, the prior digital biomarker computed from previously captured user interaction data by executing the function mapping (403). For example, biomarker comparator 312 can access prior biomarkers 303. Prior biomarkers 303 can include biomarkers generated by executing mapping function 324 on prior activity data 302. Method 400 includes comparing the digital biomarker to the prior digital biomarker (404). For example, biomarker comparator 312 can compare digital biomarker 321 to prior biomarkers 303. Method 400 includes detecting a difference between the digital biomarker and the prior digital biomarker (405). For example, biomarker comparator 312 can detect a difference between digital biomarker 321 and prior biomarkers 303.

Alternatively, or in combination, biomarker comparator 312 can access targets 323. Biomarker comparator 312 can compare digital biomarker 321 to targets 323. Biomarker comparator 312 can detect a difference between digital biomarker 321 and a target 323.

Method 400 includes forecasting a change the neuropsychological test score within a specified time range in the future based on the detected differences (406). For example, analysis module 301 can forecast mood change 322 (e.g., a depression relapse) for person 211 is to occur within a specified time range in the future (e.g., three to ten days, two to eleven days, one to twelve days, etc.). The forecast can be based on differences between digital biomarker 321 and prior biomarkers 303 and/or based on differences between digital biomarker 321 and a target 323.

Method 400 includes outputting the forecasted change (407). For example, analysis module 301 can output forecasted mood change 322. Forecasted mood change 322 can be retained at electronic device 201 and/or sent to a mental health care provider.

FIG. 3B illustrates an example computer architecture 350 that facilitates forecasting a mood change. As depicted in computer architecture 350, electronic device 201 includes durable storage 208 and network module 396. Cloud computing environment 392 includes computer 391. Computer 391 further includes storage 393 and analysis module 351. Analysis module 351 includes biomarker generator 361 and biomarker generator 362. Biomarker generator 361 and biomarker generator 362 can include functionality similar to biomarker generator 311 and biomarker generator 312 respectively.

Electronic device 201 can use network module 396 to send gesture activity 202, communication activity 203, motion activity 204, Internet activity 206, bar code/medical activities 207, and correlations 212. Computer 391 can receive gesture activity 202, communication activity 203, motion activity 204, Internet activity 206, bar code/medical activities 207, and correlations 212 from electronic device 201. Computer 391 can store gesture activity 202, communication activity 203, motion activity 204, Internet activity 206, bar code/medical activities 207, and correlations 212 in storage 393.

Prior activity data 302, other person activity 394, and prior biomarkers 383 can also be stored in storage 393. Electronic device 201 can have sent prior activity data 302 to computer 391 at an early time (e.g., a day before). Other person activity 394 can represent user interaction data received from other electronic devices and associated with one or more other persons. Prior biomarkers 383 can include biomarkers generated by executing mapping function 324 on prior activity data 302 and/or on other person activity 394.

Analysis module 351 can implement a method similar to method 400. Biomarker generator 361 can access gesture activity 202, communication activity 203, motion activity 204, Internet activity 206, bar code/medical activities 207, and correlations 212 from durable storage 208. Biomarker generator 361 can also access prior activity data 302 and/or other person activity 394. Biomarker generator 361 can execute mapping function 374 to compute digital patterns 371A . . . 371N aggregated into digital biomarker 371. Mapping function 374 can model a neuropsychological test for a brain health metric.

Biomarker comparator 362 can access prior biomarkers 383. Biomarker comparator 362 can compare digital biomarker 371 to prior biomarkers 383. Biomarker comparator 362 can detect a difference between digital biomarker 371 and prior biomarkers 383. Alternatively, or in combination, biomarker comparator 362 can access targets 373. Biomarker comparator 362 can compare digital biomarker 371 to targets 373. Biomarker comparator 362 can detect a difference between digital biomarker 371 and a target 373.

Analysis module 301 can forecast mood change 372 (e.g., a depression relapse) for person 211 is to occur within a specified time range in the future (e.g., three to ten days). The forecast can be based on differences between digital biomarker 371 and prior biomarkers 383 and/or based on differences between digital biomarker 371 and a target 373. Analysis module 301 can output forecasted mood change 372. Forecasted mood change 372 can be sent back to electronic device 201 and/or sent to a mental health care provider.

In other aspects, computer 391 (or electronic device 201) analyzes captured data against other previously captured data associated with person 211 and/or analyzes the captured data against captured data of other persons, such as, for example, other persons demographically matched to person 211. Computer 391 (or electronic device 201) can make predictions and forecasts based on captured data analysis.

Components in architectures 200, 300, and 350 can be connected to (or be part of) a network, such as, for example, a system bus, a Local Area Network (“LAN”), a Wide Area Network (“WAN”), and even the Internet. Accordingly, the components in architectures 200, 300, and 350 as well as any other connected computer systems and their components can create and exchange message related data (e.g., Internet Protocol (“IP”) datagrams and other higher layer protocols that utilize IP datagrams, such as, Transmission Control Protocol (“TCP”), Hypertext Transfer Protocol (“HTTP”), Simple Mail Transfer Protocol (“SMTP”), Simple Object Access Protocol (SOAP), etc. or using other non-datagram protocols) over the network.

In one aspect, one or more processors are configured to execute instructions (e.g., computer-readable instructions, computer-executable instructions, etc.) to perform any of a plurality of described operations. The one or more processors can access information from system memory and/or store information in system memory. The one or more processors can transform information between different formats, such as, for example, captured user interaction data, event patterns, digital patterns, digital biomarkers, forecasted mood changes, etc.

System memory can be coupled to the one or more processors and can store instructions (e.g., computer-readable instructions, computer-executable instructions, etc.) executed by the one or more processors. The system memory can also be configured to store any of a plurality of other types of data generated by the described components, such as, for example, captured user interaction data, event patterns, digital patterns, digital biomarkers, forecasted mood changes, etc.

In the above disclosure, reference has been made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general-purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, an in-dash or other vehicle computer, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. As one skilled in the art will appreciate, components may be referred to by different names. This document does not intend to distinguish between components that differ in name, but not function.

It should be noted that the sensor embodiments discussed above may comprise computer hardware, software, firmware, or any combination thereof to perform at least a portion of their functions. For example, a sensor may include computer code configured to be executed in one or more processors, and may include hardware logic/electrical circuitry controlled by the computer code. These example devices are provided herein purposes of illustration, and are not intended to be limiting. Embodiments of the present disclosure may be implemented in further types of devices, as would be known to persons skilled in the relevant art(s).

At least some embodiments of the disclosure have been directed to computer program products comprising such logic (e.g., in the form of software) stored on any computer useable medium. Such software, when executed in one or more data processing devices, causes a device to operate as described herein.

While various embodiments of the present disclosure have been described above, it should be understood that they have been presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the following claims and their equivalents. The foregoing description has been presented for the purposes of illustration and description. It is not intended to be exhaustive or to limit the disclosure to the precise form disclosed. Many modifications, variations, and combinations are possible in light of the above teaching. Further, it should be noted that any or all of the aforementioned alternate implementations may be used in any combination desired to form additional hybrid implementations of the disclosure.

What is claimed:

1. A processor implemented method comprising:
accessing user interaction data indicative of a user's interaction with a mobile device during multiple user encounters with the mobile device over a period of time, the user interaction data having been passively captured at the mobile device;
executing a function mapping to compute a digital biomarker for a brain health metric from the accessed user interaction data, the function mapping modeling a neuropsychological test for the brain health metric;
accessing a prior digital biomarker for the brain health metric, the prior digital biomarker computed from previously captured user interaction data by executing the function mapping;
comparing the digital biomarker to the prior digital biomarker;
detecting a difference between the digital biomarker and the prior digital biomarker;
forecasting a change in a score of the neuropsychological test within a specified time range in the future based on the detected differences; and
outputting the forecasted change.

2. The method of claim 1, wherein the neuropsychological test is a test for depression, anxiety, mania or psychosis

3. The method of claim 1, wherein the neuropsychological test is a test of memory, processing speed, executive function, language, dexterity or intelligence.

4. The method of claim 1, wherein accessing user interaction data comprises accessing user interaction data from the user previously diagnosed with a mental health issue.

5. The method of claim 4, wherein forecasting a change in a score of the neuropsychological test within a specified time range in the future comprises predicting a future relapse of a mental health condition.

6. The method of claim 1, wherein accessing user interaction data indicative of a user's interaction with a mobile device comprises accessing one or more of: applications opened at the mobile device, inputs typed at the mobile device, gesture patterns used on a touch screen of the mobile device, voice input received at the mobile device, accelerometer sensor data collected from the mobile device, or gyroscopic sensor data collected from the mobile device.

7. The method of claim 1, wherein forecasting a change in a score of the neuropsychological test within a specified time range in the future comprises predicting that the change is to occur between 3 and 10 days after the user interaction data was passively captured.

8. A method comprising:
accessing user interaction data indicative of a user's interaction with a mobile device during multiple user encounters with the mobile device over a period of time, the user interaction data having been passively captured at the mobile device;
executing a learning function mapping to compute a brain health metric value for a brain health metric from the accessed user interaction data, the learning function mapping modeling a neuropsychological test for the brain health metric;
accessing prior user interaction data and a corresponding prior brain health metric value for the brain health metric, the prior user interaction data indicative of previously captured user mobile device interactions, the prior brain health metric value computed from the prior user interaction data by executing the learning function mapping;
comparing the user interaction data and brain health metric value to the prior user interaction data and the prior brain health metric value;
detecting differences between one or more of: the user interaction data and the prior user interaction data or the brain health metric value and the prior brain health metric value; and
alerting a mental health provider to screen the user for a predicted clinical change based on the detected differences.

9. The method of claim 8, wherein accessing prior user interaction data and a corresponding prior brain health metric value comprises accessing prior user interaction data entered by the user at the mobile device and a prior brain health metric value for the user.

10. The method of claim 8, wherein alerting a mental health provider to screen the user for a mental health condition comprises alerting a mental health provider to screen the user for one of: depression, anxiety, mania or psychosis.

11. The method of claim 8, wherein the neuropsychological test is a test of memory, processing speed, executive function, language, dexterity or intelligence.

12. The method of claim 8, wherein alerting a mental health provider comprises alerting the mental health provider that the user is at risk of a mental health condition relapse.

13. The method of claim 12, wherein alerting a mental health provider comprises alerting the mental health provider that the user is at risk of a mental health condition relapse within a specified time period in the future.

14. A computer system comprising:
a processor;
system memory coupled to the processor and storing instructions configured to cause the processor to:
access user interaction data indicative of a user's interaction with a mobile device during multiple user encounters with the mobile device over a period of time, the user interaction data having been passively captured at the mobile device;
execute a function mapping to compute a digital biomarker for a brain health metric from the accessed user interaction data, the function mapping modeling a neuropsychological test for the brain health metric;
access a prior digital biomarker for the brain health metric, the prior digital biomarker computed from previously captured user interaction data by executing the function mapping;
compare the digital biomarker to the prior digital biomarker;
detect a difference between the digital biomarker and the prior digital biomarker;
forecast a change in a score of the neuropsychological test within a specified time range in the future based on the detected differences; and
output the forecasted change.

15. The computer system of claim 14, wherein instructions configured to cause the processor to access user interaction data comprise instructions configured to cause the processor to access user interaction data from the user previously diagnosed with a mental health issue.

16. The computer system of claim 14, wherein instructions configured to cause the processor to forecast a change in a score of the neuropsychological test within a specified time range in the future comprise instructions configured to cause the processor to predict a future relapse of a mental health condition.

17. The computer system of claim 14, wherein instructions configured to cause the processor to accessing user interaction data indicative of a user's interaction with a mobile device comprises instructions configured to cause the processor to access one or more of: applications opened at the mobile device, inputs typed at the mobile device, gesture patterns used on a touch screen of the mobile device, voice input received at the mobile device, accelerometer sensor data collected from the mobile device, or gyroscopic sensor data collected from the mobile device.

18. The computer system of claim 14, wherein instructions configured to cause the processor to forecast a change in a score of the neuropsychological test within a specified time range in the future comprise instructions configured to cause the processor to predict that the change is to occur between 3 and 10 days after the user interaction data was passively captured.
